(19) United States
(12) Patent Application Publication
(10) Pub. No.: US 2004/0137550 A1
(43) Pub. Date: Jul. 15, 2004

(54) Acetylcholinesterase assay

(76) Inventors: Han Lim Lee, Kuala Lumpur (MY); Nazni Wasi Ahmad, Kuala Lumpur (MY); Sa?apos;Diyah Ibrahim, Kuala Lumpur (MY)

Correspondence Address: Omri M. Behr, 325 Pierson Ave, Edison, NJ, 08837-3123, US

(21) Appl. No.: 10/744,162

(22) Filed: Dec. 22, 2003

(30) Foreign Application Priority Data
Dec. 18, 2002 (MY) PI 20024748

(51) Int. Cl. C12Q 1/44

(52) U.S. Cl. 435/20

(57) ABSTRACT

A method for the assay of acetylcholinesterase in a sample of saliva is provided. The method can be used for the detection of poisoning caused by carbamate insecticide or organic phosphorus-based agricultural chemicals in a patient. A kit using the method of the invention is also provided.

### FIELD OF THE INVENTION

The present invention relates to a saliva-based assay for the detection of acetylcholinesterase (AChE) in humans.

### BACKGROUND OF THE INVENTION

Cholinesterase (ChE) is a generic term used to describe a group of related enzymes that hydrolyse choline esters. Two types of cholinesterase exist in the body that differs in terms of enzymological properties, physiological function and distribution in the body. The first is acetylcholinesterase (also referred to as true cholinesterase) which specifically breaks down acetylcholine, exists in a large amount in erythrocytes (red blood cells), neural tissue, muscle and so forth, and is distributed in relation to these physiological functions. The other is butylcholinesterase (also referred to as pseudocholinesterase), which acts on cholines, such as benzoylcholine and butylcholine, exists in a large amount in the serum and liver, is produced in the liver, and the physiological action of which is considered to most likely be involved with the neuralmuscular system.

Both true cholinesterase and pseudocholinesterase are responsible for the hydrolysis of acetylcholine (ACh) released at the nerve endings, which is necessary to depolarise the nerve so that it can be repolarised in the next conduction event. This rapid depolarisation and repolarisation process is propagated along the electrically excitable membrane thus mediating transmission of nerve impulses.

At present, the cholinesterase that is frequently measured in clinical laboratory examinations is cholinesterase in serum. This enzyme is a glycoprotein having a molecular weight of approximately 340,000 and is composed of four identical subunits. Each subunit is composed of 574 amino acids and has nine asparagine-coupled carbohydrate chains. Clinically, a decrease in its activity, as determined by measuring this enzyme, has significance in terms of determining the degree of functional impairment to liver parenchyma in liver disease, and particularly chronic liver parenchymal disorders such as liver cirrhosis and chronic hepatitis. Since serum cholinesterase is produced in liver parenchymal cells, its decrease indicates a chronic functional decrease of liver cells. In addition, acute decreases in cholinesterase activity are observed in cases of poisoning by organic phosphorus-based agricultural chemicals or carbamate insecticides, measurement of the activity of this enzyme is indispensable in these cases. In addition, increases in the activity of this enzyme are observed prominently in nephritic syndromes.

In the past, measurement of cholinesterase was performed by various methods including a thiocholine method, wherein thiocholine released by cholinesterase is measured by colouring it with an SH group assay reagent using the synthetic substrates of acetylthiocholine, propionylthiocholine and butylthiocholine; a UV method wherein a direct decrease in substrate is measured in the form of the reduction in absorbance of the ultraviolet using benzylcholine as the substrate; a pH colorimetric method wherein an organic acid produced by cholinesterase is measured using a pH indicator; and an enzyme method (cholinoxidase method) wherein the hydrogen peroxide produced during specific decomposition of choline by cholinesterase is measured with a coloration system using benzoylcholine as the substrate and cholinoxidase and peroxidase as cooperative enzymes.

All the methods described above are based on blood (see for example, U.S. Pat. No. 6,461,831, published on 8 Oct. 2002) or ocular fluid (see for example, U.S. Pat. No. 5,595,883, published on 21 Jan. 1997) investigations of cholinesterase activity in the human body. Both are invasive methods and cause a considerable inconvenience and burden on the test subjects. The requirement that blood sample be extracted from the test subjects for investigations of cholinesterase activity in the human body have discouraged many people suspected of poisoning caused by organic phosphorus-based agricultural chemicals or carbamate insecticide from attending screening programmes to detect changes in the cholinesterase activity. In addition, screening programmes using blood-based methods to detect cholinesterase activity in the human body can only be carried out on a scheduled basis, either monthly or half-yearly because of the costs involved. The requirement of a trained person to extract blood samples required and in conducting laboratory tests means that the test subjects need to be present in person at the clinics. Where the test subjects involved are daily paid workers, this means stopping work and a loss of much required subsistence income for them. It would therefore be desirous if there could be devised a simple and affordable low cost procedure wherein the general public could carry out the tests at any time on their own without requiring supervision of a trained personnel. It would also be desirous if the method is non invasive.

We have now discovered that saliva instead of blood can be used in detecting cholinesterase activity in the human body. In particular the invention relates to the detection of acetylcholinesterase enzyme in saliva obtain from humans, thus overcoming the cumbersome process of extracting blood from test subjects.

Improper applications or handlings of agrochemicals expose farm and public health workers to the risk of chemical poisoning. Indiscriminate application of insecticides before harvesting to protect crops against pests may leave unacceptably high level of residual chemicals that endanger public health. In developing countries where organic farming is expensive and not prevalent, organically grown foods are restricted to the upper echelon of the society. There is therefore a need for a user-friendly and an affordable rapid screening test kit to be developed for use in screening chemical poisoning. This invention can be adapted as a test kit for use in the detection of changes in cholinesterase activity in the human body suspected of poisoning caused by organic phosphorus-based agricultural chemicals or carbamate insecticides.

### Reference List

The present specification refers to the following publications, each of which is expressly incorporated herein by reference.

1. Ammon, R. (1933). Die fermentative Spaltung des Acetylcholine. Pflugers Archive Ges Physiology 233:286-291.

2. Crane, C. R., Sanders, D. C. and Abbot, J. N. (1975). Cholinesterase use and interpretation of cholinesterase measurements. In: Sunshine I, eds. Methodology for analytical toxicology. CRC Press.

3. Dass, P., Mejia, M and Landes, M. (1994). Check sample. Clinical Chemistry 34: 135-158.

4. Ellman, G. L., Courtney. K. D., Andres Jr. V. and Featherstone, R. M. (1961). A new and rapid colorimetric determination of acetylcholinesterase activity. Biochemical Pharmacology 7: 88-95.

5. Howard, J., East, N. and Chaney, J. (1978). Plasma cholinesterase activity in early pregnancy. Archive of Environmental Health 33:277-279.

6. Kaplan, A. L. and Pesce, A. J. (1996). Clinical Chemistry, Theory, Analysis and Correlation. 3^{rd}. Edition. (Ed.: Kazmierczak, S. C.) pp. 967-968. Mosby.

7. Kaplan, E. and Tildon, J. T. (1963). Changes in red cell enzyme activity in relation to red cell survival in infancy. Pedaetrics 32:371-375.

8. Mayne, P. D. (1994). Clinical Chemistry in Diagnosis and Treatment. 6^{th} Edition. Pp. 311. Edward Arnold Publication.

9. Moss, D. W. and Henderson, M. B. (1994). Tietz Textbook of Clinical Chemistry. 2^{nd} Edition. (Eds.: Burtis, C. A., Ashwood, E. R.) pp. 877-882. W B Saunders Co.

10. Ravin, H. A., Tsou, K. C. and Seligman, A. M. (1951). Colorimetric estimation and histochemical demonstration of serum cholinesterase. Journal of Biological Chemistry 191:843-857.

11. Wilkinson, J. H. (1976). The principles and Practice of Diagnostics Enzymology. 1^{st} Edition. Pp. 119-120. Edward Arnold Publication.

### SUMMARY OF THE INVENTION

It is an object of the present invention to provide a new diagnostic assay for the early detection of an increase or decrease in acetylcholinesterase activity in humans, which assay overcomes the aforesaid limitations of the prior art methods.

Accordingly, in its broadest aspect the invention provides a saliva-based method for the early detection of change in acetylcholinesterase activity in humans.

To accomplish the object, the Ellman's method was used to detect the activity of acetylcholinesterase enzyme through micro assay of human saliva. A change in the optical density is defined as an increase or a decrease in the quantity of the enzyme compared to the average levels of the enzyme in the unaffected control population.

The method of the invention is preferably applicable to the detection of change in acetylcholinesterase activity in humans under the influence of carbamate insecticides or organic phosphorus-based agricultural chemicals poisoning.

The second object of the present invention is to develop a test kit incorporating the invention described above for use as a screening tool for early detection of carbamate insecticides or organic phosphorus-based agricultural chemicals poisoning in workers working in places where there is heavy application of carbamate insecticides or organic phosphorus-based agricultural chemicals. The test kit could also be used by the general public who may be affected by consumption of harvested crops with high level of residual insecticides due to indiscriminate application of insecticides by poorly educated farmers before harvesting.

To accomplish the second object, there is provided a test kit comprising six components and uses a micro assay method described above to detect a change in acetylcholinesterase activity in a sample of non-centrifuged human saliva. A change in the enzyme activity is scored visually by comparing the colour obtained in the test with a standard colour chart.

### DETAILED DESCRIPTION OF THE INVENTION

In this experiment, the Ellman's method was used to detect the activity of acetylcholinesterase in human saliva through a macro assay and a micro assay.

### EXAMPLE 1

Macro Assay of Enzyme Activity

The macro assay involved a sample size of 12 test subjects consisting of 6 females and 6 males. The test subjects were selected according to the method of Moss et al (1994). All the 12 test subjects were healthy adults and were not at risk of having their body cholinesterase levels altered: e.g. long term exposure to insecticide usage, suffering from acute infections and chronic disease affecting heart, lungs, kidney, brain or the endocrine system, genetic disorders, psychiatric states or have undergone any recent surgical procedures.

Saliva samples were obtained from the 12 test subjects and filter papers were used to remove the bubbles and debris such as sputum. The 12 saliva samples were kept cold in an ice box. The samples were transferred by a plastic tip pipette into plastic vials and centrifuged at 8,000 rpm for 10 minutes at a temperature of 4° C. In the mean time, a potassium phosphate buffer solution of pH 6.8 was prepared. The buffer solution consists of 0.4735 g Na₂HPO₄ in 50 ml of distilled water and 0.454 g KH₂PO₄ in 50 ml of distilled water. The substrate, acetylthiocholine iodide solution, kept at 4° C. was prepared using 0.0375 g acetylthiocholine iodide, 5 ml acetone and 45 ml potassium phosphate buffer. The substrate solution was mixed in a bottle and the bottle was covered with aluminium foil to prevent any exposure to light. After that, the coupling agent known as the Ellman's solution was prepared using 0.0065 g of 5,5-dithiobis (2-nitrobenzoic acid) (DTNB) and 50 ml of potassium phosphate buffer. The reagent bottle containing the Ellman's solution was also covered by aluminium foil to prevent exposure to light.

About 333 μl of each of the 12 saliva samples were pipetted into twelve 1 ml curvettes respectively. Then about 333 μl of acetone-buffer solution of acetylthiocholine iodide (substrate) was added. This was followed by the addition of 333 μl of DTNB into each respective curvette. In addition, a blank was prepared using 333 μl of substrate, 333 μl of DTNB and 333 μl of phosphate buffer, without saliva. The optical density of the blank was 0.08. The 12 curvettes were incubated at room temperature (25° C.) for 30 minutes. After 30 minutes, the intensity of the chromophore produced by DTNB was scored visually and also by using a spectrophotometer at 410 nm. The optical density of each curvette was recorded. The results are shown in Table 1.

### EXAMPLE 2

Micro Assay of Enzyme Activity

The experiment was repeated by using a micro assay sample. The same procedure for the macro assay of acetylcholinesterase was followed except for the different in amount. Instead of using 333 μl each of potassium buffer, substrate, saliva and DTNB, a smaller amount, 50 μl of each reagent was used. Saliva samples were obtained from the same 12 test subjects. First, a blank was prepared by pipetting 50 μl of phosphate buffer into 50 μl of substrate and 50 μl of DTNB and placed in a well of a microtitration plate. The optical density of the blank was 0.08. 50 μl of substrate and 50 μl of DTNB were pipetted into 12 other wells in the microtitration plate. Then, 50 μl of centrifuged saliva was pipetted into each individual well and incubated at room temperature (25° C.) for 30 minutes. After 30 minutes, the optical density was read using the immunoassay reader at a wavelength of 410 nm. The results are shown in Table 3. By this method, it was found that the optical densities for all the 12 samples were similar to the ‘blank’.

The experiment was repeated by using saliva that was not centrifuged. The saliva was collected and stored in microcentrifuge tubes at 4° C. and left for one hour for sedimentation to occur. It is noted that cholinesterase is a comparatively stable enzyme and can be stored for several weeks either frozen or at 0-5° C. (Wilkinson, 1976). After one hour, the procedure of pipetting 50 μl of substrate, DTNB and saliva into the wells of the microtitration plate was repeated. The optical densities were read by the immunoassay reader at a wavelength of 410 nm. The results are shown in Table 4.

Results

The results showed that there is a significant amount of acetylcholinesterase in human centrifuged saliva for the macro assay and non-centrifuged saliva for the micro-assay. T-test on the macro assay (Table 2) and micro assay (Table 5) was used to obtain statistical results. By pooling the results of both female and male test subjects, the mean optical density was 0.15±0.04 for the macro assay and 0.34±0.19 for the micro assay. From the T-test results it was also concluded that there was no sex-linked significance in salivary acetylcholinesterase activity since p>0.05 is obtained for both macro assay (Table 2) and micro assay (Table 5).

The results also showed that without centrifugation, a significant amount of acetylcholinesterase activity was detected even with the small amount of 50 μl of saliva used. This is partly due to the fact that cholinesterases have a very high molecular weight, in the range of 2-12×10⁶ dalton (Wilkinson, 1976). Thus, with such a small amount, the acetylcholinesterase would have been centrifuged down. Furthermore, although acetylcholinesterase is a soluble enzyme, much of the enzyme protein adheres to particulate matter and it is therefore undesirable to clarify homogenates by centrifugation prior to determination of cholinesterase activity (Wilkinson, 1976).

By comparing the optical density of both macro and micro assay, it was observed that the micro assay of saliva without centrifugation had a greater optical density than the macro assay of saliva with centrifugation. A higher yellow intensity produces a greater optical density reading and this reflects a higher acetylcholinesterase activity. This is consistent with the observation of Moss et al. (1994). Therefore, detection of acetylcholinesterase activity in the human's saliva is best done using a micro assay of saliva that has not been centrifuged.

TABLE 1
Acetylcholinesterase levels in test subjects by sex
through macro assay

| | Optical Density (OD) |
|---|---|
| Females | |
| F1 | 0.154 |
| F2 | 0.171 |
| F3 | 0.166 |
| F4 | 0.107 |
| F5 | 0.114 |
| F6 | 0.252 |
| Males | |
| M1 | 0.119 |
| M2 | 0.160 |
| M3 | 0.126 |
| M4 | 0.113 |
| M5 | 0.109 |
| M6 | 0.157 |

TABLE 2
Comparative Acetylcholinesterase levels in male and
female test subjects

| | Sample | | | |
|---|---|---|---|---|
| | Size | Mean | Std. Dev. | SEM |
| Females | 6 | 0.16 | 0.05 | 0.02 |
| Males | 6 | 0.13 | 0.02 | 0.01 |

95% Confidence Interval for Difference: 0.02 to 0.08
T = 1.296 with 10 degrees of freedom; p = 0.224

TABLE 3
Acetylcholinesterase levels in centrifuged saliva as
determined by micro assay

| | Optical Density (OD) |
|---|---|
| Females | |
| F1 | 0.060 |
| F2 | 0.073 |
| F3 | 0.333 |
| F4 | 0.062 |
| F5 | 0.082 |
| F6 | 0.079 |
| Males | |
| M1 | 0.069 |
| M2 | 0.095 |
| M3 | 0.087 |
| M4 | 0.064 |
| M5 | 0.076 |
| M6 | 0.108 |

TABLE 4
Acetylcholinesterase levels in non-centrifuged saliva
as determined by micro assay

| | Optical Density (OD) |
|---|---|
| Females | |
| F1 | 0.396 |
| F2 | 0.236 |
| F3 | 0.411 |
| F4 | 0.426 |
| F5 | 0.490 |
| F6 | 0.203 |
| Males | |
| M1 | 0.165 |
| M2 | 0.324 |
| M3 | 0.236 |
| M4 | 0.235 |
| M5 | 0.128 |
| M6 | 0.838 |

TABLE 5
Comparative Acetylcholinesterase activity in
non-centrifuged saliva in female and male test subjects

| | Sample | | | |
|---|---|---|---|---|
| | Size | Mean | Std. Dev. | SEM |
| Females | 6 | 0.36 | 0.11 | 0.05 |
| Males | 6 | 0.32 | 0.26 | 0.11 |

95% Confidence Interval for Difference: 0.22 to 0.30
T = 0.337 with 10 degrees of freedom; p = 0.743

Development of a Test Kit

The present invention can also be adapted into a test kit for use in the detection of acetylcholinesterase activity in humans suspected of carbamate insecticides or organic phosphorus-based agricultural chemicals poisoning. Test kits made according to the present invention comprises six components. Each of the component is kept in a different container, such as for example a dark coloured reagent bottle as follows:

Bottle A: Potassium phosphate buffer 10 ml.

Bottle B: Acetone 1 ml.

Bottle C: Acetylthiocholine iodide 7.5 mg.

Bottle D: Potassium phosphate buffer 9 ml.

Bottle E: Potassium phosphate buffer 10 ml.

Bottle F: DTNB 1.3 mg.

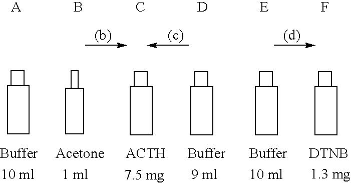

Test kits made according to the present invention consisting essentially of a potassium phosphate buffer (0.05M, pH 6.8), acetylthiocholine iodide substrate and 5,5 dithiobis (2-nitrobenzoic acid) (DTNB) as the coupling agent may be employed according to the protocol described below:

(a) Bottle A is a homogenizing buffer.

(b) Pour B into C and completely dissolve C by mixing.

(c) Pour D into C and mix well.

(d) Pour E into F and mix well to dissolve the substance.

(e) The working solutions are: solutions A, C, and F.

(f) Collect fresh saliva by spitting into a clean paper cup.

(g) Pipette 50 μl of saliva into an eppendorf tube and top up to 0.5 ml marking with solution A.

(h) Put one drop of the saliva from (g) into each well of microtiter plate using pasteur pipette (8-10 replicates).

(i) Drop one drop of solution C into the first 2 wells.

(j) Then drop one drop solution F into these two wells.

(k) Incubate at room temperature for 30 minutes.

(l) Score by eye by comparing the colour obtained in the test with a colour chart provided in the kit.

(m) If the yellow colour obtained from the reaction is lighter than that provided in the colour chart, the level of acetylcholinesterase present in the saliva is considered low and the test subject would be screened out for more thorough tests.

A low acetylcholinesterase in the sample saliva indicates that the test subject may be suffering from carbamate insecticides or organic phosphorus-based agricultural chemicals poisoning. The test subject may then be referred to a hospital for a more thorough confirmatory test and to determine the level of poisoning that has taken place.

To ensure proper working of the kits, instructions, either as inserts or labels, including quantities of the components to be used and guidelines for mixing the reagents may be included in the kits.

While the invention has been described in terms of the embodiments shown above, the skilled artisan will appreciate that various modifications, substitutions, omissions, and changes may be made without departing from the spirit thereof. Accordingly, it is intended that the scope of the present invention be defined by the following claims, including equivalents thereof.

1. A method for the assay of acetylcholinesterase in a sample of saliva, which method comprises:
(a) obtaining a sample of saliva from a test subject;
(b) mixing the sample with acetone-buffer solution of acetylthiocholine iodide;
(c) adding 5,5 dithiobis (2-nitrobenzoic acid) (DTNB) to the solution obtained in step (b);
(d) incubating the contents in (c) above at room temperature for 30 minutes;
(e) scoring the intensity of the colour produced by DTNB visually; and
(f) comparing the colour obtained in (e) with a colour chart.

2. A method as claimed in claim 1 wherein the intensity of the colour produced by DTNB is scored using a spectrophotometer at 410 nm.

3. Use of the method as claimed in claims 1 for the detection of poisoning caused by carbamate insecticide or organic phosphorus-based agricultural chemicals in a test subject, which method comprises:
(a) obtaining a sample of saliva from a test subject;
(b) mixing the sample with acetone-buffer solution of acetylthiocholine iodide;
(c) adding 5,5 dithiobis (2-nitrobenzoic acid) (DTNB) to the solution obtained in step (b);
(d) incubating the contents in (c) above at room temperature for 30 minutes;
(e) scoring the intensity of the colour produced by DTNB visually; and
(f) comparing the colour obtained in (e) with a colour chart.

4. A method as claimed in claim 3 wherein the intensity of the colour produced by DTNB is scored using a spectrophotometer at 410 nm.

5. A test kit for the assay of acetylcholinesterase in a sample of saliva using the method as claimed in claim 1, said kit comprises the following components:
Bottle A: Potassium phosphate buffer 10 ml.
Bottle B: Acetone 1 ml.
Bottle C: Acetylthiocholine iodide 7.5 mg.
Bottle D: Potassium phosphate buffer 9 ml.
Bottle E: Potassium phosphate buffer 10 ml.
Bottle F: DTNB 1.3 mg.
characterized in that the said test kit is employed as follows:
(a) Bottle A is a homogenizing buffer.
(b) Pour B into C and completely dissolve C by mixing.
(c) Pour D into C and mix well.
(d) Pour E into F and mix well to dissolve the substance.
(e) The working solutions are: solutions A, C, and F.
(f) Collect fresh saliva by spitting into a clean paper cup.
(g) Pipette 50 μl of saliva into an eppendorf tube and top up to 0.5 ml marking with solution A.
(h) Put one drop of the saliva from (g) into each well of microtiter plate using pasteur pipette (8-10 replicates).
(i) Drop one drop of solution C into the first 2 wells.
(j) Then drop one drop solution F into these two wells.
(k) Incubate at room temperature for 30 minutes.
(l) Score by eye by comparing the colour obtained in the test with a colour chart.

6. Use of the test kit as claimed in claim 5 for the detection of poisoning in test subjects caused by carbamate insecticides or organic phosphorus-based agricultural chemicals.
